(19) United States
(12) Patent Application Publication
(10) Pub. No.: US 2009/0326501 A1
(43) Pub. Date: Dec. 31, 2009

(54) SOPHISTICATED DISPOSABLE DIAPER

(76) Inventors: Diane E. Foley, Denton, TX (US); Elmer J. Haggadone, Flower Mound, TX (US)

Correspondence Address: Michael A. O'Neil. P.C., 8150 North Central Expressway, Suite 1150, DALLAS, TX, 75206, US

(21) Appl. No.: 12/491,040

(22) Filed: Jul. 22, 2009

Related U.S. Application Data
(60) Provisional application No. 61/075,636, filed on Jun. 25, 2008.

(51) Int. Cl. A61F 13/551; A61F 13/84

(52) U.S. Cl. 604/385.06

(57) ABSTRACT

A disposable diaper is provided with a pouch which contains a moisture-laden flexible sheet for use in cleaning an infant or toddler after the disposable diaper has received bodily waste. A plastic bag that initially contains the disposable diaper is also used to receive the disposable diaper following use. Alternatively, a plastic bag for receiving the used disposable diaper is initially contained in a second pouch mounted on the disposable diaper.

This application claims the benefit of U.S. Provisional Application Ser. No. 61/075,636 filed on Jun. 25, 2008, the contents of which are hereby incorporated by reference herein.

### DESCRIPTION OF THE DRAWINGS

FIG. 1 illustrates a front-view of a young child wearing a first embodiment of a sophisticated disposable diaper wherein a pouch and flap are sealed;

FIG. 2 illustrates the front-view of a young child wearing the first embodiment of the sophisticated disposable diaper wherein the pouch and flap are unsealed and a saniwipe® or similar material is being removed from the pouch;

FIG. 3 illustrates the first embodiment of the sophisticated disposable diaper sealed inside a plastic bag ready for use;

FIG. 4 illustrates the first embodiment of the sophisticated disposable diaper as shown in FIG. 3 but with the sophisticated disposable diaper being removed from the plastic bag in preparation for use;

FIG. 5 illustrates disposing in the plastic bag of FIG. 3 the first embodiment of the sophisticated disposable diaper after having been soiled;

FIG. 6 illustrates a second embodiment of the sophisticated disposable diaper wherein a sealed pocket containing a self-disposing plastic bag is adjacent to and below a sealed pouch; and

FIG. 7 illustrates the second embodiment of the sophisticated disposable diaper wherein the pocket containing the self-disposing plastic bag is unsealed and the plastic bag is encasing the sophisticated disposable diaper.

### DESCRIPTION OF THE INVENTION

As many caregivers can attest, the clean-up and disposition of a young child's soiled disposable diaper can be a messy, grungy, and pungent undertaking. Cleaning and disinfecting the soiled areas of the child's skin as well as the child's surrounding environment requires the caregiver to maintain a constant supply of disinfecting agents that are utilized each time the disposable diaper is removed from the child. Once the soiled disposable diaper is removed, it is typically placed in the rubbish where an astringent odor usually emanates from the soiled disposable diaper to the surrounding vicinity.

More specifically when the young child soils the diaper, the caregiver locates a damp cloth, towelette, or similar sanitary-type of material to clean and disinfect the surface area of the child's skin that was in contact with the soiled disposable diaper. Since a young child typically soils a diaper several times a day, the caregiver utilizes a damp cloth, towelette, or similar sanitary-type material several times a day. As such, the caregiver keeps a steady supply of sanitary-type material on hand for use and lugs the sanitary-type material from place to place when traveling.

Once a soiled disposable diaper is disjoined from the young child, the caregiver typically discards the soiled disposable diaper in the rubbish. Since the soiled disposable diaper is not sealed in a container or bag, it typically exudes a pungent odor around the vicinity of disposal, causing irritation and discomfort to those nearby.

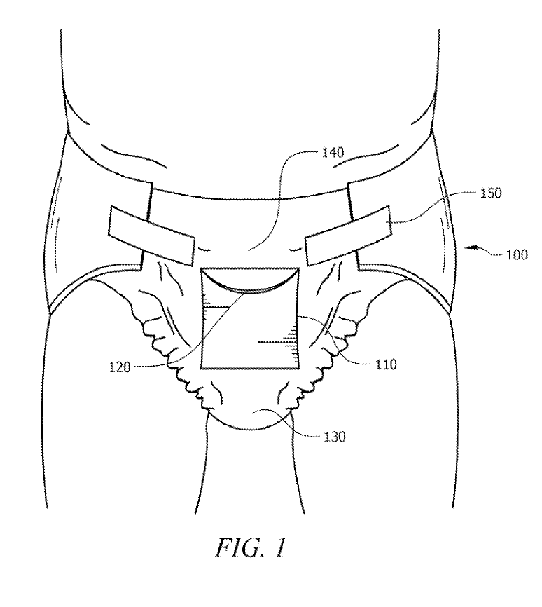

FIG. 1 shows a young child wearing one embodiment of a sophisticated disposable diaper 100. One such embodiment comprises a pouch 110 and a flap 120 attached to the sophisticated disposable diaper 100. The flap 120, positioned at an opening end of the pouch 110, seals the contents of the pouch 110 until torn by a caregiver.

For optimal use, the pouch 110 is attached to a front area of the sophisticated disposable diaper 100 wherein the front area is defined by the front orientation of a young child. However the pouch 110 can also be attached to a back area of the sophisticated disposable diaper 100 wherein the back area is defined by the back orientation of a young child.

The pouch 110 extends from a crotch area 130 to a top area 140 wherein the top area 140 is defined below an elastic band 150 or other fastening and/or securing mechanism of the sophisticated disposable diaper 100. At the top area 140, the flap 120 seals the inner contents of the pouch 110 until torn by a caregiver.

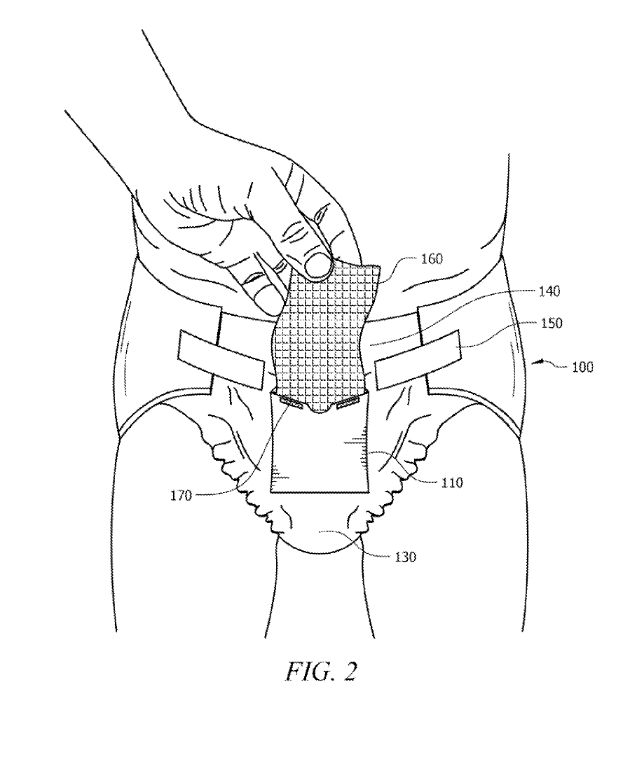

As illustrated in FIG. 2, a sheet of disposable saniwipe® 160 or similar material used to clean, sanitize, and disinfect the areas of a young child's skin along with affected nearby surroundings is contained in the pouch 110. The disposable saniwipe® 160 or similar material is sealed in the pouch 110 by the flap 120, locking in moisture and preventing the saniwipe® 160 or similar material from dehydrating.

An adhesive material 170 seals the flap 120 to the pouch 110. The adhesive material 170, such as but not limited to a polymer compound, secures the flap 120 to the pouch 110 until a caregiver breaks the adhesion by tearing the flap 120.

Once the adhesion is broken and the flap 120 is upraised, the disposable saniwipe® 160 or similar material is removed from the pouch 110 as shown in FIG. 2. Once removed, the disposable saniwipe® 160 or similar material is utilized to clean and disinfect.

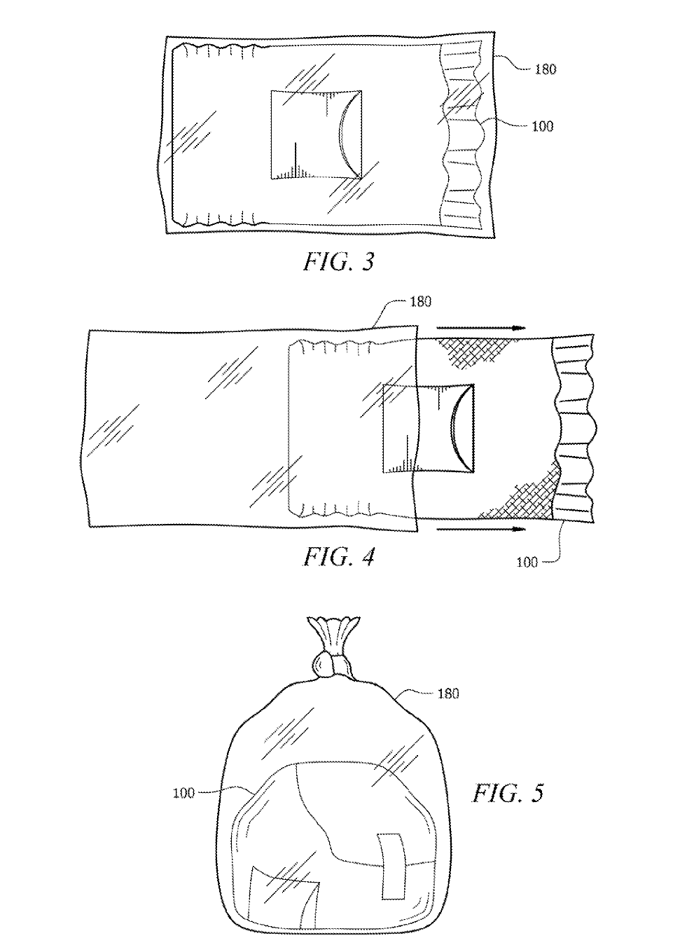

As shown in FIG. 3, the first embodiment of the sophisticated disposable diaper 100 provides for a plastic bag 180 that fully encloses the sophisticated disposable diaper 100 at the time of purchase. Upon use, the caregiver opens the plastic bag 180, removes the sophisticated disposable diaper 100 as shown in FIG. 4, and affixes the sophisticated disposable diaper 100 to the young child.

Once the young child has soiled the sophisticated disposable diaper 100 and the caregiver has removed the saniwipe® 160 or similar material from the pouch 110 as shown in FIG. 2, the caregiver detaches the sophisticated disposable diaper 100 from the young child and inserts it into the plastic bag 180 as shown in FIG. 5.

As illustrated in FIG. 5, the sophisticated disposable diaper 100 is placed in the plastic bag 180 and sealed via a tie, knot, or other method to prevent pungent odors from emanating from the plastic bag 180. As such, the sophisticated disposable diaper 100 is discarded without causing odorous irritation and/or discomfort to persons nearby.

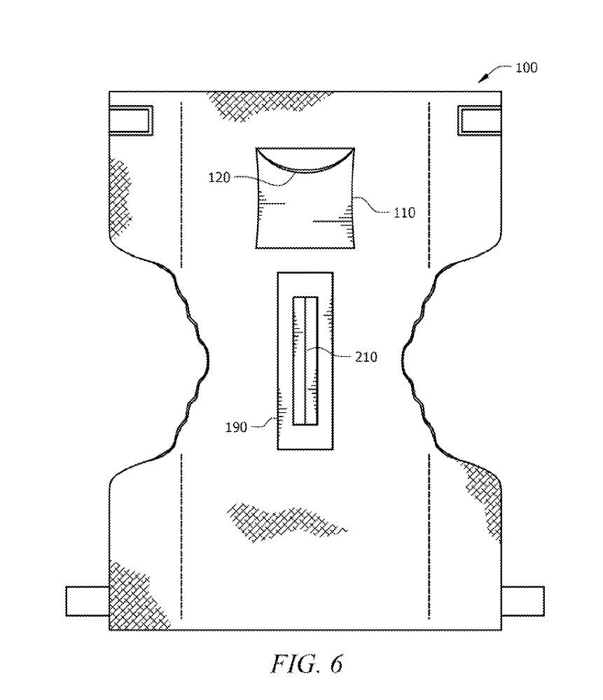

As illustrated in FIG. 6, a second embodiment of the sophisticated disposable diaper comprises a pocket 190 adjacent to but below the pouch 110. Within the pocket 190 is a self-disposing plastic bag 200 utilized to encase the sophisticated disposable diaper 100 after having been soiled. The pocket 190 containing the self-disposing plastic bag 200 is sealed to prevent a young child from gaining access to the self-disposing plastic bag 200.

The sealed area 210 of the pocket 190 is covered with a polymeric-type material. To utilize the self-disposing plastic bag 200 to encase the sophisticated disposable diaper 100, the polymeric-type material covering the sealed area 210 of the pocket 190 is removed by tearing or cutting. Once the sealed area 210 is torn or cut, the self-disposing plastic bag 200 is accessible.

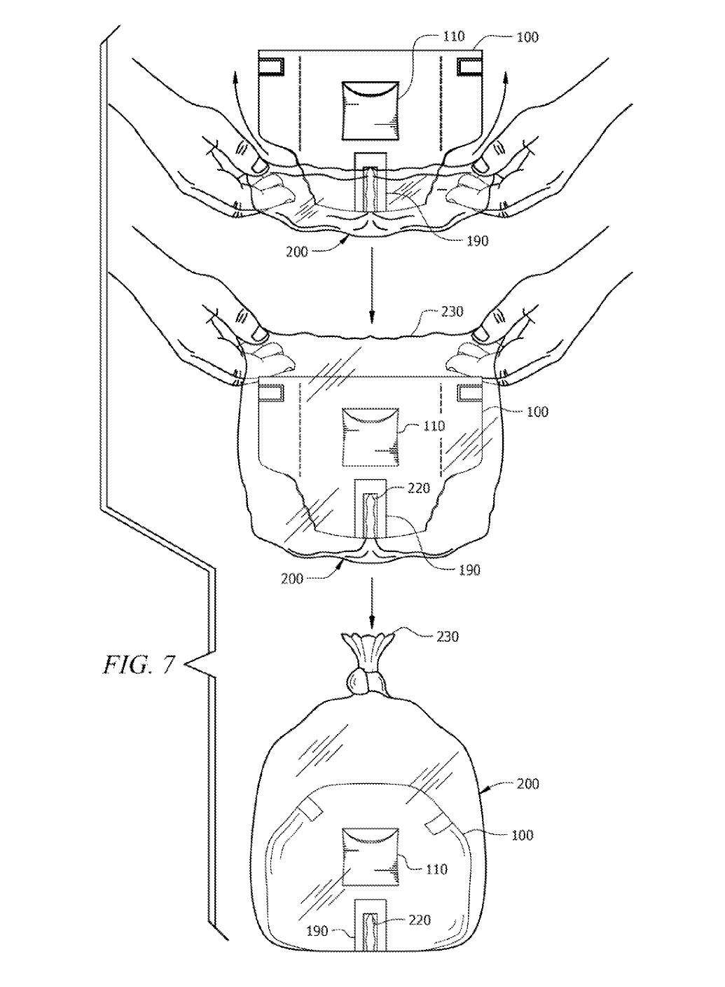

As illustrated in FIG. 7, the self-disposing plastic bag 200 comprises a closed end 220 and an opened end 230. The closed end 220 is attached to the pocket 190 of the sophisticated disposable diaper 100. When the pocket 190 is unsealed, the opened end 220 of the self-disposing plastic bag 200 is pulled from the pocket 190 until fully extended.

Once fully extended, the opened end 230 of the self-disposing plastic bag 200 is positioned around the sophisticated disposable diaper 100. After encasing the sophisticated disposable diaper 100, the opened end 230 of the self-disposing plastic bag 200 is sealed via a tie, knot, or other method to prevent pungent odors from emanating to the surrounding vicinity. Then, the self-disposing plastic bag 200 encasing the sophisticated disposable diaper 100 is discarded into the rubbish.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

We claim:

1. For use in conjunction with disposable diapers of the type worn by babies and toddlers to receive body waste, the improvement comprising:
a normally sealed pouch mounted on the disposable diaper;
a moisture-laden flexible sheet for cleaning the wearer of the diaper after bodily waste has been received by the diaper;
the moisture laden flexible sheet being initially stored in the normally sealed pouch whereby moisture is retained in the flexible sheet pending utilization thereof; and
a flexible plastic bag for receiving the disposable diaper after it has received bodily waste thereby facilitating appropriate disposal of the disposable diaper.

2. The improvement according to claim 1, wherein the flexible plastic bag initially receives the disposable diaper prior to utilization thereof and subsequently receives the disposable diaper after it has received bodily waste.

3. The improvement according to Clam 1 further including a second pouch mounted on the disposable diaper for initially containing the plastic bag;
the plastic bag being secured to the disposable diaper at a location within the second pouch; and
the plastic bag being extendable from the second pouch for receiving the disposable diaper following utilization thereof.
